(19) United States
(12) Patent Application Publication
(10) Pub. No.: US 2001/0032000 A1
(43) Pub. Date: Oct. 18, 2001

(54) Combined aromatherapy air diffuser with lonizer

(76) Inventor: Simon Dotan, Netanya (IL)

Correspondence Address: Home Care Technologies Ltd, Att: SIMON DOTAN, 45 Pinchas Rossen St., TEL-AVIV, 69512, IL

(21) Appl. No.: 09/729,277

(22) Filed: Dec. 5, 2000

Related U.S. Application Data
(60) Provisional application No. 60/179,161, filed on Jan. 31, 2000.

(51) Int. Cl. A61F 7/00; A61F 7/12

(52) U.S. Cl. 607/96

(57) ABSTRACT

A device, combining the prominent Aromatherapy treatment, together with ionizing.

The system creates combined molecules of etheric oil and O₃, loaded with static electrical charge.

The procedure is done by airflow, carrying molecules of various kinds of etheric oils.

The loaded airflow, with etheric oil molecules, passes by a metal grid, which creates O₃ molecules at its edges. The O₃ molecules are gravitated to the etheric oil molecules that flow nearby, and creating a new molecule. These new molecules are loaded with a static electrical charge, being formed from the connection with the O₃ molecule.

LED system, having the homogenous wavelength, is installed in the front side of the device, having a focus at 50-mm. approx. in order to heat up the treated area, and by this to allow a better penetration of the etheric oil.

### BACKGROUND AND SUMMARY OF THE INVENTION

This invention relates to a device that actually operates in three different modes, usually done by a separate device for each mode of action. Said proposed device not only that it does these three separate modes of action, but also, due to the fact that it does the air diffusing prior to the ionization, it causes the air stream that contains inside it the free etheric oil molecules, to gather with the free O₃ molecules that are created throughout the way, by the ionization system.

Transferring the static charge to the etheric oil molecules intensifies their chemical reaction, while being in contact with any living tissue whatsoever. The device additional uniqueness is that later on, once it is in contact with the living tissue, this tissue has already been pre-heated by the focused lamps system, which is located in the front side of the device. This system stimulates the blood circulation in the tissue, so by the time the loaded air stream arrives; its absorption in the tissue is much more effectively.

### DETAILED DESCRIPTION OF THE DRAWINGS

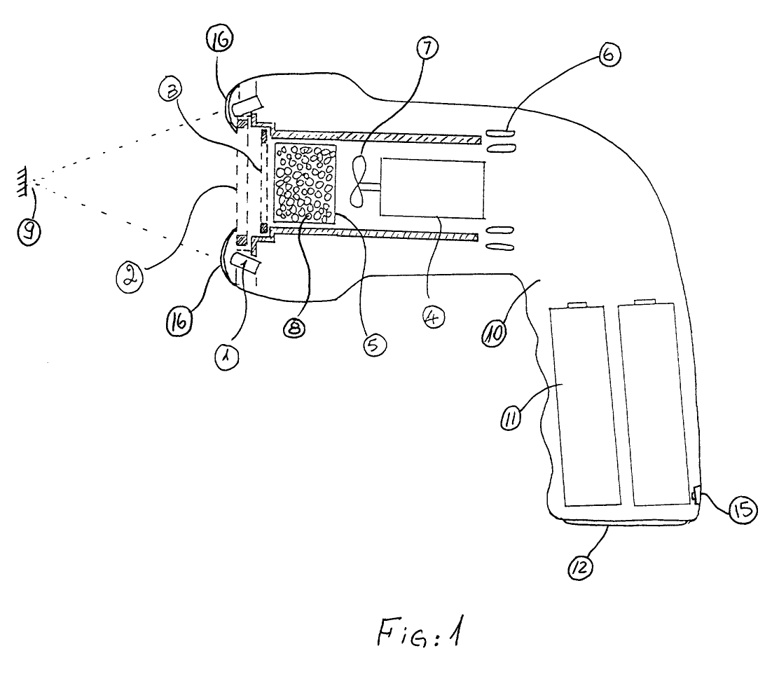

FIG. 1 Describes the product in section that includes details of the units, oils container, fan motor, ionization systems and LED system, and of course the energy source- either batteries or an external electrical source by plug.

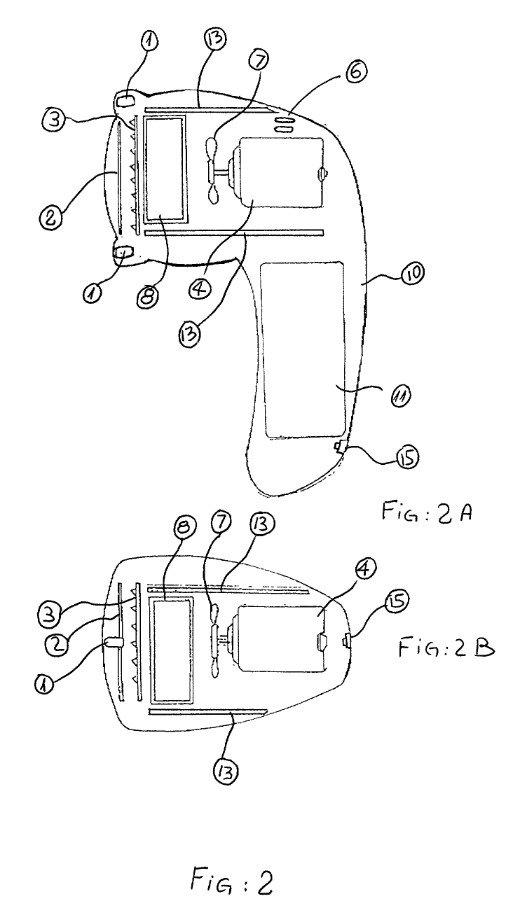

FIG. 2 Describes in section the location. FIG-2A describes a side section; FIG-2B describes an upper section, which also includes location of the important parts.

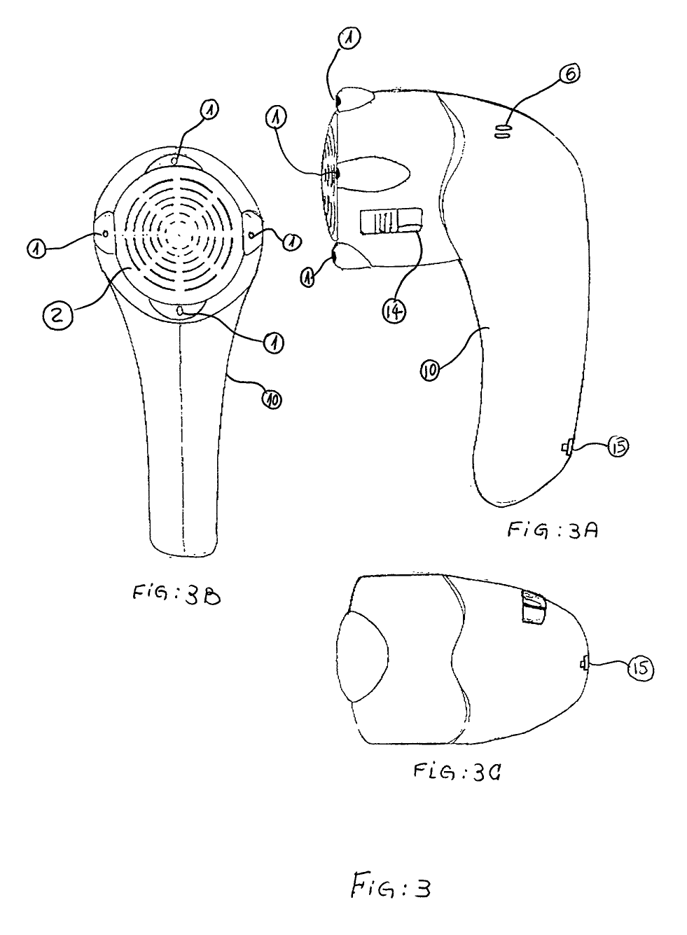

FIG. 3 Generally describes the proposed device projection, in three dimensions.

### DETAILED DESCRIPTION OF THE PREFERRED EMBODIMENT

As illustrated in FIG. 1, said invention is a combination of three different devices. Motor 4, along with fan 7, constitute an air diffuser, which transmits air through container 5, which is either filled with aromatic beads, or with a material saturated with liquid etheric oil 8. The air imputes to the device are located behind motor 4 and are openings 6 The air stream that passes through container 5, continues its way and pas nearby a ionization system with grid 3, out of which the air stream continues its way to the front side 2 of said device, thus preventing the user to be in direct contact with the electrical charge area, located nearby grid 3.

LED light system, having a coherent wavelength, such as LED 1, is pointed towards a focusing point located in the front side of said device, around 50 mm. at point 9. Each lamp has an optic lens, lens 16, for concentrating the light beam.

Said device has a carrying lever, in which the energy source, 11, is located inside the unit. External input source for supplying alternative energy via socket 15. Battery door for replacement of battery 12.

What is claimed is:

1. Said invention is supposed to provide the user with three integrated elements of alternative medicine treatment. The uniqueness of said device is reflected in the order of its modes of operation as it determines the procedure.
The first one—A directed etheric oils molecules stream, of any kind of etheric oils, by passing through a special container of etheric oils in beads or other liquid form.
The second—a system that creates O₃ molecules. The etheric oil molecules, flow nearby the ionization system, the two molecules are attracted to each other by gravity. The result of it is; the new molecules are charges with electrical charge given by the O₃. The connection between the O₃ molecules and the etheric oil molecules is done automatically and creates a very active new molecule.
The third—serial of lamps, having a constant and coherent wavelength, being focused to a specific point at the front side of said device. Said focus point is located in the center of the flowing excel of the loaded air stream, with the charged new molecules. The focus point heats the focus area and thus rises up the blood circulation in this area.

2. Said invention of claim 1, wherein said airflow system is located inside a tunnel construction, and is done by a fan, pointing the airflow to the front side of the device. The fan motor can be adjusted to the desired speed, for allowing maximal absorption of O₃ molecules by the etheric oil molecules.

3. Said invention of claim 1, wherein the air stream penetrates into a special container, containing etheric oil in various forms, either in beads or liquid, absorbed within a sponge base; the air flow releases the molecules of the etheric oil and leads them with it toward the next station.

4. Said invention of claim 1, where in a stream, saturated with etheric oil molecules continues its way and passes within a grid that creates an ionization of the air, while simultaneously, the O₃ molecules are cached to the etheric oil molecules that diffuse nearby and physically connect to those molecules, by gravitation.

5. Said invention of claim 1, whereas in the front side of the devise the lamps are gathered together towards a focusing point, located at a constant distance, for example: 50 mm. away from the safety mash.

6. Said invention of claim 1, wherein the air flow loaded with the etheric oil molecules that have passed through the ionization system, have obtained the electrical charge while connecting with the O₃ molecules, and continues its way through the safety mash, until the focusing point.

7. Operating any one of its three elements separately, by pressing a single push-button can, also operate the said invention of claim 1.
